(19) United States
(12) Patent Application Publication
(10) Pub. No.: US 2023/0270839 A1
(43) Pub. Date: Aug. 31, 2023

(54) VACCINE COMPOSITIONS

(71) Applicant: Medi-Immune Limited, Staffordshire (GB)

(72) Inventors: John Paul James, Staffordshire (GB); James Prince Love, Staffordshire (GB); David John Maudsley, Staffordshire (GB)

(21) Appl. No.: 18/142,697

(22) Filed: May 3, 2023

Related U.S. Application Data
(63) Continuation of application No. 16/751,635, filed on Jan. 24, 2020, now abandoned.
Application No. 16/751,635 is a continuation of application No. PCT/EP2018/069669, filed on Jul. 19, 2018.

(30) Foreign Application Priority Data
Jul. 27, 2017 (GB) GB 1712098.1
Mar. 31, 2018 (GB) GB 1805393.4

(51) Int. Cl. A61K 39/145; A61K 9/00

(52) U.S. Cl. CPC A61K 39/145; A61K 9/0078; A61K 2039/544

(57) ABSTRACT

The invention relates to vaccines for the prevention or treatment of infectious diseases, and to methods of preparing or delivering such vaccines. In particular, a vaccine for use in the prevention or treatment of disease is used in a dose of, via a parenteral route without adjuvant less than 0.03 µg antigen and with adjuvant less than 0.003 µg antigen, and via a mucosal route without adjuvant less than 1 µg antigen and/or the equivalent of 1.6 × 10⁷ PFU and with adjuvant less than 0.04 µg antigen and/or the equivalent of 1.6 × 10⁷ PFU

### CROSS-REFERENCE

This application is a continuation of PCT Application No. PCT/EP2018/069669, filed Jul. 19, 2018, which claims priority to GB 1712098.1, filed Jul. 27, 2017, and GB 1805393.4, filed Mar. 31, 2018, which are incorporated herein by reference in their entirety.

### FIELD OF INVENTION

The invention relates to vaccines for the prevention or treatment of infectious diseases, and to methods of preparing or delivering such vaccines.

### BACKGROUND OF THE INVENTION

### Infectious Diseases

Infectious diseases arguably pose the greatest threat to health there is. The World Health Organisation (WHO) lists influenza and tuberculosis (TB) as the two greatest current infectious threats to world health with both of these being airborne pathogens. Other threats include emerging infections such as Middle East Respiratory Syndrome virus (MERS), which is also transmitted by the airborne route. In animals, one serious pathogen has been foot and mouth disease virus (FMDV) in cattle. Again this is a pathogen transmitted from one individual to another by the airborne route, potentially over distances of up to several miles.

Key developments in the history of protection from infectious diseases include vaccines and antibiotics. In 1796 Edward Jenner demonstrated the effectiveness of vaccination, forming a scientific basis for future vaccination programmes. Vaccination may have saved more lives than any other medical intervention apart from perhaps antibiotics. In 1928 Alexander Fleming isolated penicillin from the mould Penicillium notatum, which eventually led to the development of antibiotics for the treatment of bacterial infections. The effectiveness of antibiotics is reduced however by the increased incidence of antibiotic resistance in bacteria. Clearly antibiotics have had a beneficial impact in relation to bacterial infections but not directly on infections caused by viruses which are not susceptible to antibiotics. A third important development has been public health and hygiene measures, such as clean water, hand washing and using handkerchiefs, which have prevented the transmission of infections and saved countless lives.

Although both treatment and prevention reduce the harms caused by infectious agents, clearly it is better to prevent infection in the first place if possible rather than treat it. There are essentially two ways of protecting people from infection: interfering with the route of transmission and reducing the susceptible population. This is true for endemic infections, as well as for epidemics and pandemics.

Globalisation has an effect on both the transmission of pathogens and the availability of a pool of susceptible individuals. Global travel means that pathogens can travel round the world more easily. It also makes the pool of susceptible people larger. As a consequence, any new infectious disease, arising for example by mutation of an animal pathogen (e.g. MERS, SARS, bird flu, Ebola, HIV etc.), has the opportunity to spread to susceptible individuals, and to be maintained within the human population in the long term. Fortunately, pathogens typically become less severe with time due to the selection of variants that are better at being transmitted in preference to those that kill their host quickly.

### Other Health Effects of Infection

Infections, whether acute or chronic, cause inflammation which has a range of effects on health. For example, inflammation is thought to be responsible for many aspects of ageing from cardiovascular disease to dementia. Infection is also a key cause of exacerbation of diseases of old age including diabetes and chronic obstructive pulmonary disease (COPD). The influenza vaccine is one of the most cost effective ways of “treating” people with these diseases (e.g. Hovden, A.-O., Cox, R.J., and Haaheim, L.R., 2007, Int J COPD 2, 229-240; Centers for Disease Control and Prevention: “People at High Risk of Developing Flu-Related Complications”, and “Adults With Chronic Conditions: Get Vaccinated”.

### Vaccines

As a result of Jenner’s demonstration of the effectiveness of cow pox/vaccinia in protecting people from smallpox a WHO-led campaign resulted in the elimination of smallpox as a pathogen in the wild in 1979. Smallpox along with Rinderpest, poliovirus type 2, and possibly poliovirus type 3, remain the only infections eliminated by vaccination so far. Of note among the vaccines currently available are the vaccines against influenza. Seasonal influenza accounts for approximately 3000 deaths a year in the US. In a good year, i.e. when the vaccine matches the antigenicity of the current circulating influenza strain, vaccination reduces symptomatic influenza A infection by 60% with a consequent reduction in hospitalisations and mortality of 60% for those vaccinated (Centers for Disease Control and Prevention: “Vaccine Effectiveness - How Well Does the Flu Vaccine Work?”, and “CDC Presents Updated Estimates of Flu Vaccine Effectiveness for the 2014-2015 Season.

### SUMMARY OF THE INVENTION

The present invention is based on the experimental finding that low doses (“mini doses”) of vaccine provided immune protection against subsequent exposure to the live infectious pathogen. Immune protection was provided at doses lower and significantly lower than those previously used for injection vaccinations or aerosol/mucosal vaccines. Doses of 500 PFU provided immune protection, i.e. surprisingly, doses less than 10⁷ PFU were effective with for example influenza. Protection was provided without the need for an adjuvant.

Thus, there is provided a vaccine for use in the prevention or treatment of infection wherein the dose is a “mini dose” or a low dose, typically below or significantly below the dose known or predicted to be effective based on public knowledge at the time of filing this application. For example, less than one half of the dose used for intra nasal immunisation, or less than one tenth of the dose used for intra muscular immunisation, or less than a hundredth of the dose used for intra muscular immunisation. Typically, the dose is less than 0.03 µg of antigen, such as less than 0.003 µg of antigen and/or less than the equivalent of 10⁷ PFU, less than 1 × 10⁷ PFU or less then the equivalent of 1.6 × 10⁷ PFU, and typically it is delivered without an adjuvant. The vaccine may be delivered mucosally, e.g. to the digestive tract, intra-nasally (i.n.), by the intra-pulmonary route (i.pul.), to the genital tract, to the urinary tract or to the eye. The vaccine may be delivered to the respiratory tract or the respiratory system. The vaccine may be delivered to the lungs by the intra-pulmonary route (i.pul.).

In some embodiments of the invention repeated doses may be delivered. In some embodiments whole inactivated pathogen may be used. Two, three or more doses may be delivered i.pul.

Furthermore, the vaccine may be made in situ using a protective respiratory device that inactivates the pathogen in the air. The low dose of the invention allows use of the protective respiratory device in everyday situations to protect and immunise against airborne infections.

The invention therefore relates to the prevention or treatment of disease by using a vaccine, wherein the dose of the vaccine is a “mini-dose”. For example, the vaccine may be administered in a dose of less than 0.04 µg, or a dose equivalent to 1.6 × 10⁷ PFU. Where the vaccine is administered via a parenteral route, the dose of vaccine may be less than 0.03 µg without adjuvant. Where the vaccine is administered via parenteral route, the dose of vaccine may be less than 0.003 µg antigen with adjuvant. Where the vaccine is administered via a mucosal route without adjuvant, the dose may be less than 1 µg antigen and/or the equivalent of 1.6 × 10⁷ PFU. Where the vaccine is administered via a mucosal route and with adjuvant the dose may be less than 0.04 µg antigen and/or the equivalent of 1.6 × 10⁷ PFU. Thus, in some embodiments, the vaccine comprises or is delivered with an adjuvant. In other embodiments, the vaccine does not comprise or is not co-administered with an adjuvant.

In any of these embodiments, the dose may be less than 0.003 µg and/or less than the equivalent of 10⁷ PFU. In any of these embodiments, the dose may be less than 0.001 µg and/or less than the equivalent of 5 × 10⁶ PFU. In any of these embodiments, the dose may be less than 0.0003 µg and/or less than the equivalent of 10⁶ PFU.

Preferably, the dose is an effective dose. The dose may be greater than 3 × 10⁻⁸ µg and/or greater than the equivalent of 10 PFU. The method may prevent or treat an infectious disease in an individual, wherein the method comprises administering an effective amount of a vaccine to the individual, wherein the effective amount is less than 3 × 10⁻² µg antigen and/or the equivalent of 10⁷ PFU.

The disease to be treated or prevented may be a respiratory infection and/or airborne infection, thus the vaccine may be against a respiratory infection and/or airborne infection. The disease may be influenza, tuberculosis, MERS, SARS, rhinovirus, measles, Ebola, Chlamydia pneumonia, respiratory syncytial virus, pneumococci or FMDV. In a typical embodiment, the disease is influenza. The vaccine may be a viral vaccine or a bacterial vaccine. The vaccine may be used in the treatment of heterologous (related) and/or heterologous (unrelated) pathogen.

In any of these embodiments, the vaccine may be administered to the lungs. In any of these embodiments, the vaccine may be delivered as and/or comprises an aerosol. In any of these embodiments, the vaccine may be inactivated vaccine.

In any of these embodiments, the vaccine may be a pre-manufactured vaccine preparation, or may be made in situ. Where the vaccine is made in situ, it may be made in situ using a portable device and/or an air sterilisation device. For example, the subject to be treated may be the wearer of the protective device and/or in the environment of the wearer of the protective device.

In some embodiments, the subject to be treated has already been primed by exposure to the same, or similar, pathogen and/or has been vaccinated with a vaccine for the same or similar pathogen.

The vaccine may be delivered in one or more administrations. The vaccine may be delivered in an initial priming dose followed by a boost or followed by more than one boost. The vaccine may be delivered on 3 or more occasions to a subject.

In a particular embodiment as described herein, an effective amount of the inactivated, typically synthetic, recombinant, killed or non-replication competent, vaccine is delivered by aerosol to the lungs on 3 or more occasions at a dose of less than the equivalent of 10⁶ PFU (inactivated) or less than 0.001 µg.

In some embodiments, the dose is between the equivalent of 10 PFU (inactivated) and 10⁷ PFU (inactivated), the vaccine is delivered by intra pulmonary administration as an aerosol, the vaccine is delivered on more than 2 occasions, and the vaccine is a made in situ using a portable device and/or the vaccine is a pre-manufactured vaccine preparation.

The vaccine may be provided in combination with a nebuliser. A composition comprising a bacterium or virus may be provided in combination with a device such as a protective device, comprising a disinfection chamber in fluid communication with a face mask wherein the chamber is arranged to disinfect and/or sterilise the fluid containing the bacterium or virus in the chamber prior to discharge of the fluid from the chamber, and wherein the dose of vaccine is less than 0.03 µg or less than 0.003 µg and/or less than the equivalent of 10⁷ PFU or less than the equivalent of 1.6 × 10⁷ PFU. For example, the chamber may be arranged to inactivate the virus in the chamber prior to discharge of the composition from the chamber into the face mask, such that the device delivers a dose of inactivated virus of less than 0.003 µg and/or less than the equivalent of 1.6 × 10⁷ PFU into the face mask.

### DETAILED DESCRIPTION OF THE INVENTION

### Therapy and Prophylaxis

The present invention relates to methods of preventing and treating infectious diseases, and to vaccine compositions and devices that are used in such methods. Where methods and uses are described here it is to be understood that they relate to a method of treatment/prevention, a product for use in a method of treatment/prevention and/or use of a product in the manufacture of a medicament for treatment/prevention.

In general, a vaccine is a preparation that provides immunity, such as active acquired immunity, to a particular disease. The vaccine typically comprises an agent that resembles a disease-causing pathogen, such as an agent that can trigger an immune response that recognises the pathogen. The vaccine may comprise a weakened, inactivated or killed form of the pathogen. The vaccine may comprise one or more proteins or other molecules that are present in or on the pathogen. Typically, the vaccine induces a response from a subject’s immune system, allowing the subject to produce an immune response against any future contact with the pathogen.

The vaccines of the invention can be used in a method of treatment by therapy. For example, a vaccine of the invention may be administered as a primary prophylactic agent to a subject at risk of infection by a pathogen or exposure to an antigen, or can be used as a secondary agent for treating subjects who are already infected.

A vaccine for use in accordance with the present invention is capable of inducing a protective immune response in the subject to whom it is administered. The immune response may be induced against any undesirable infectious agent or suitable antigen, such as an antigen that is present on or in a pathogen. The vaccine may therefore be a vaccine against a pathogen, such as a vaccine against a virus, bacterium, fungus, other prokaryotic or eukaryotic cell or organism, or against any protein, glycoprotein or other molecule or structure that can be used to target any such pathogen. For example, in the case of the influenza virus, the vaccine may be against haemagglutinin (HA), a glycoprotein found on the surface of influenza viruses. A vaccine may target a single antigen or may target multiple antigens, such as two or more antigens from a single pathogen, or two or more antigens from two or more pathogens.

Typically, a vaccine as described herein is used in a method of treatment, such as a method of treatment by therapy or prophylaxis to prevent or treat disease, in particular infectious disease. Typically, the disease is caused by a pathogen, e.g. a bacterial or viral infection The infection may be caused by an airborne pathogen. Typically, the infection is a respiratory infection. Typically, the infection is caused by an airborne virus, more typically an RNA virus, more typically a negative strand RNA virus, more typically an orthomyxovirus, more typically an influenza virus, and more typically an influenza A virus. The infection may be caused by an influenza virus such as influenza A, influenza B or influenza C. Typically, the virus is from the papovavirus, adenovirus, herpesvirus, poxvirus, parvovirus and/or hepadnavirus family. More typically the virus is from the picornavirus, astrovirus, togavirus, arenavirus, bunyavirus, retrovirus, rhabdovirus, filovirus, reovirus and/or bimvirus family. More typically the virus is from the orthomyxovirus, paramyxovirus and/or coronavirus family; these three families are all similar being enveloped RNA viruses, with paramyxoviruses and coronaviruses being slightly larger than orthomyxoviruses.

The infection may be a respiratory infection such as influenza, parainfluenza, MERS, SARS, rhinovirus or respiratory syncytial virus.

The infection may be a pandemic infection, such as pandemic influenza, MERS, SARS or other similar infection. Typically, the vaccine is against a pathogen that has newly emerged and/or for which there is no conventional or other vaccine available.

Doses of vaccine in terms of particle numbers and/or infectious units (inactivated) should therefore deliver similar immunising effects as influenza virus (an orthomyxovirus). Both orthomyxoviruses and paramyxoviruses are negative strand RNA viruses.

Where the infection is a bacterial infection, typically the bacterium is from the mycobacteria or mycoplasma families.

Typically, the vaccine is against influenza, tuberculosis, MERS, SARS, Ebola, pneumococci or FMDV. Typically, the vaccine is against measles, parainfluenza, respiratory syncytial virus, and/or a rhinovirus. Typically, the vaccine is against, influenza, rhinovirus or respiratory syncytial virus.

The vaccine of the invention may also be used against infections of other mucosal sites such as Salmonella, cholera, Helicobacter pylori, Legionella, and/or HIV.

As discussed below, doses of influenza virus of 500 PFU (equivalent to 1.5 × 10⁻⁶ µg HA antigen) were effective at generating protective immunity in ferrets. This dose is therefore immunogenic against influenza A (see example 1 below). Clearly, similar doses of similar viruses will be equally immunising. This includes other influenza A viruses, other members of the orthomyxovirus family, typically influenza B, especially as they are treated similarly in conventional vaccines. Clearly other virus families, such as paramyxoviruses, will require similar amounts of antigen.

The vaccine may prevent or treat infection with the pathogen. The vaccine may prevent, reduce or ameliorate one or more symptoms normally associated with infection with that pathogen. The vaccine may prevent, reduce or ameliorate one or more symptoms or conditions that are associated with or caused by infection with the pathogen. For example, the vaccine may prevent or treat inflammation associated with the infection, and/or symptoms or conditions associated with such inflammation such as diabetes and COPD.

Typically, the vaccine will be used to treat an animal. The animal may be a bird, typically the bird is poultry, typically the bird is a chicken. The animal may be a mammal, including a non-human mammal; more typically the vaccine will be used to treat a human. The vaccine may be used in a medical or veterinary context. The subject may be male or female and may be an infant, a child or an adult. Because the agents in some of these compositions are inactivated, they are particularly well suited for administration to “at risk individuals” such as the elderly, children, or infected or unwell persons.

The subject to be treated may be immunologically naive with respect to the pathogen being treated. Typically, the subject and/or population to be treated is primed by exposure to the same or similar pathogen and/or antigen and/or by vaccination.

### Vaccine Preparations

A vaccine for use in accordance with the present invention may be provided as a vaccine composition. A suitable vaccine composition may be any composition capable of inducing a protective immune response in the subject to whom it is administered. The immune response may be induced against any undesirable infectious agent or suitable antigen, such as an antigen that is present on or in a pathogen. The vaccine may therefore be a vaccine against a pathogen, such as a vaccine against a virus, bacterium, fungus, other prokaryotic or eukaryotic cell or organism, or against any protein, glycoprotein or other molecule or structure that can be used to target any such pathogen. A vaccine may target a single antigen or may target multiple antigens, such as two or more antigens from a single pathogen, or two or more antigens from two or more pathogens. The vaccine may target one or more strains of a pathogen.

A vaccine may comprise one or more active therapeutic components, such as viral, peptide, protein based, cell-based and/or nucleic acid based products, such as live viral vaccines, live bacterial vaccines, killed or inactivated viral or bacterial vaccines, vectors encoding an antigen of interest.

The vaccine may further comprise one or more pharmaceutically acceptable diluents, excipients, carriers and/or adjuvants. One exemplary pharmaceutically acceptable carrier is physiological saline. Other physiologically acceptable carriers and their formulations are known to those skilled in the art and examples are described, for example, in Remington’s Pharmaceutical Sciences, (18^{th} 15 edition), ed. A. Gennaro, 1990, Mack Publishing Company, Easton, PA incorporated herein by reference. Thus, the invention provides compositions for mucosal or parenteral administration that include the above-mentioned agents dissolved or suspended in an acceptable carrier, typically an aqueous carrier, e.g., water, buffered water, saline, PBS, and the like.

The vaccine may contain pharmaceutically acceptable auxiliary substances as required to approximate physiological conditions, such as pH adjusting and buffering agents, tonicity adjusting agents, wetting agents, detergents and the like. For example, the vaccine may be a composition comprising one or more stabilisers (e.g. serum albumin proteins, human or bovine serum albumin, gelatin, casein, PVP or combinations or any thereof), bulking agents (e.g. sugars such as lactose, sucrose, fructose, sugar alcohols such as mannitol or sorbitol, or combinations of any thereof), buffers (e.g. amino acids, histidine, glutamic acid alkali metal salts) etc.

An adjuvant is a material that when used in conjunction with a vaccine antigen enhances the immune response to the vaccine antigen. Vaccine adjuvants improve the body’s immune response and often allow for smaller amounts of an inactivated virus or bacterium to be used in a vaccine. Where adjuvants are used, they may be selected based on the route of administration or based on the particular vaccine or the pathogen to be vaccinated against. Adjuvants include alum, AS03, GPI00100, saponin, ISCOMATRIX, Freunds complete and incomplete adjuvants; cytokines may also be used as adjuvants as can Toll-Like-Receptor agonists.

In the case of mucosal administration, chitin microparticles (CMP) can be used (Asahi-Ozaki et al., Microbes and Infection 8:2706-2714, 2006; Ozdemir et al., Clinical and Experimental Allergy 36:960-968, 2006; Strong et al., Clinical and Experimental Allergy 32: 1794-1800, 2002). Other adjuvants suitable for use in administration via the mucosal route include the heat-labile toxin of E.coli (LT) or mutant derivatives thereof. In the case of inactivated virus, parenteral adjuvants can be used including, for example, aluminum compounds (e.g., an aluminum hydroxide, aluminum phosphate, or aluminum hydroxyphosphate compound), liposomal formulations, synthetic adjuvants, such as (e.g., QS21), muramyl dipeptide, monophosphoryl lipid A or polyphosphazine.

Squalene or squalene based adjuvants such as MF59 or AS03 have been used in influenza vaccines. Adjuvants based on inulin, a natural plant-derived polysaccharide, such as Advax™ have been used in influenza vaccines. Such adjuvants may be used in vaccines according to the invention, particularly vaccines against airborne or respiratory pathogens such as influenza vaccines.

The vaccines of the invention, whether for intra-pulmonary delivery or for delivery via any other route described herein may be used with adjuvant. A vaccine composition of the invention may comprise one or more adjuvants. A vaccine or a vaccine composition of the invention may be administered in combination with one or more adjuvants, such as the adjuvants described herein.

Typically, the vaccine is used without the need for an adjuvant. Thus, a vaccine composition may comprise no adjuvants and/or may be administered without any adjuvant.

There are a number of situations where changes in dosage, or addition of adjuvants have been tested, including in different ages and in obesity. For example, older adults have lower antibody responses to influenza vaccine. There is some evidence that in this population high-dose influenza vaccine (60 µg of HA per strain compared with 15 µg per strain) is more effective (Wilkinson K et al., 2017, Vaccine 35 (21) 2775-2780). Obesity is a complicating factor and vaccinated obese animals and adults have decreased neutralising antibody responses. Squaline-based adjuvant AS03 or alum enhanced the immune response, increasing both neutralising and non-neutralising antibody levels. However, obese mice were not protected against influenza virus challenge even with this increase in antibody levels (Karlsson EA, Hertz T et al., 2016, MBio 7(4) e01144-16). Several clinical trials have applied adjuvants to influenza vaccine to allow dose-sparing. Gordon et al., 2016, (Vaccine 34, 3780-6) found in humans that a reduced dose of 15 µg seasonal trivalent inactivated influenza vaccine (TIV, 5 µg per strain) when administered with Advax adjuvant elicited a comparable immune response to standard trivalent influenza vaccine alone (45 µg, 15 µg per strain) as measured by haemagglutination inhibition assays. Poder et al. (2014, Vaccine, 32, 1121-9) found in children that for vaccines adjuvanted with AS05 reduced doses of HA of 3.75 µg or 1.9 µg delivered once or twice elicited comparable or better HAI antibody responses than non-adjuvanted vaccine. Similarly, Langley et al., (2012, Pediatr Infect Dis J 31 (8) 848-58) found in children that, for 2 doses 21 days apart, AS03 adjuvanted H1N1 vaccine at doses of 3.75 µg and 1.9 µg gave antibody responses comparable or better than unadjuvanted vaccine at 7.5 or 15 µg per dose. All vaccines apart from the non-adjuvanted 7.5 µg HA vaccine met European regulatory criteria.

The vaccine compositions may be provided as a solid, a liquid, a freeze-dried or lyophilised form or an aerosol. The vaccine may be dissolved in a physiologically compatible solution or buffer, such as a pharmaceutically acceptable carrier as described herein.

Prior to administration, a vaccine may be provided or may be prepared by methods known in the art. Standard methods of preparation and formulation can be used as described, for example, in Remington’s Pharmaceutical Sciences (18th edition), ed. A. Gennaro, 1990, Mack Publishing Company, Easton, PA.

Vaccines are usually manufactured in advance and prepared so that known doses can be delivered to individual subjects. Such vaccine preparations that are manufactured in advance and are provided for subsequent administration to a subject are defined as “manufactured vaccine preparations” or “pre-manufactured vaccine preparations” in this application. Such preparations may be provided in a sealed and/or sterilised container allowing for the withdrawal of a dose of the vaccine for administration to the subject. Such preparations may be provided in a multi-use container allowing for the removal of one or more doses of the vaccine for administration to a subject, for example where a course of administration includes more than one administration of the vaccine, or for administration of one or more doses to one or more different subjects.

Alternatively, as discussed further below, a vaccine may be produced in situ for administration to a subject. For example, a vaccine preparation may be produced in situ using a respiratory device that inactivates or kills a pathogen, thereby delivering the inactivated pathogen to the user as a vaccine preparation.

### Vaccine Administration

A vaccine for use in the present invention may be delivered to the subject via any suitable route. By administration” or “administering” or “delivery” is meant a method of giving a dosage of a vaccine composition of the invention to a subject (e.g., a human), where the route of administration is, e.g., intranasal, intrapulmonary, topical, systemic, inhalation, oral, intravenous, subcutaneous, intravascular, intra-arterial, intraperitoneal, intraventricular, nasal, or intramuscular. Typically, the compositions are administered via the intrapulmonary route (e.g. via aerosol inhalation), parenterally (e.g., by intramuscular, subcutaneous, or intravenous injection), or by oral ingestion, or by topical application.

The preferred route of administration can vary depending on various factors, e.g., the components of the pharmaceutical composition, and the site of the potential or actual disease. Vaccines are typically delivered by injection. This can be, for example, by a parenteral route such as the subcutaneous (s.c.), intramuscular (i.m.) or intra dermal routes. The invention also contemplates the administration of vaccines via a mucosal route, such as via intranasal delivery, oral, or intra-pulmonary delivery. For example, live attenuated measles vaccine has been delivered into the lungs: intra pulmonary delivery (Patent application WO2007/000308, Vaccine nebulisers, Cutts F et al.). A mucosal route of administration such as intrapulmonary administration may be used. A mucosal route of administration such as intrapulmonary administration may be used where the vaccine is used to treat or prevent a respiratory and/or airborne infection. The vaccine may therefore be delivered to the lungs, i.e. intra pulmonary immunisation (i.pul. immunisation). The i.pul. immunisation may be delivered in the form of an aerosol.

Delivery to the lungs and intra pulmonary administration typically involve delivery to the lower lung, not just intra tracheal and intra bronchial delivery but typically delivery to the bronchioli and more typically to the alveoli of the lung.

Typically, the vaccine is for use in therapy. Typically, the vaccine is for use in the prevention or treatment of an infection or an infectious disease. Typically, the vaccine is administered by a parenteral route (e.g. i.m., s.c., intra dermally and/or via a patch) and/or via mucosal route (e.g. i.n., i.pul.). More typically the vaccine is delivered via a mucosal route. More typically the vaccine is delivered via the lungs, the i.pul. route. Where the dose is delivered via a patch and/or in combination with an adjuvant typically the dose is less than 0.003 µg antigen. Where the dose of vaccine is delivered to the lungs without an adjuvant typically the dose is less than 0.03 µg antigen.

Typically, the vaccine is for use in therapy and the vaccine is delivered by aerosol to the lungs on 3 or more occasions at a dose of less than 10⁷ PFU (inactivated) or less than 0.01 µg. In addition, typically an effective amount of vaccine is delivered.

### Vaccine Dosage

The present invention also provides compositions that include prophylactically or therapeutically effective amounts of one or more vaccines, as described herein. The compositions containing an effective amount of vaccine can be administered for prophylactic and/or therapeutic treatments. In prophylactic applications, vaccines of the invention may be administered to the subject (e.g., a human) in an amount sufficient to delay, reduce, or prevent the onset of clinical or subclinical disease. In therapeutic applications, vaccines are administered to a patient (e.g., a human) already suffering from a disease, such as infection with a pathogen, in an amount sufficient to cure or at least partially arrest the symptoms of the condition and its complications.

An amount adequate to accomplish this purpose is defined as a “therapeutically effective dose.” Determination of an appropriate dosage amount and regimen can readily be determined by those of skill in the art based on their knowledge and the teaching herein. Amounts effective for this use may depend on the severity of the disease or condition and the weight and general state of the patient.

For the purposes of this application the concept of “mini dose” of vaccine is used. This is a dose of vaccine that is less, or substantially less, than that previously known, or predicted, to be an effective dose of vaccine via the specified route in that species for that microorganism. In particular, doses that are not known or predicted to provide protective immunity when delivered by the intra pulmonary route are mini doses, and are one embodiment of the current invention.

The doses described herein may refer to the amount of the active ingredient in a vaccine composition, such as the amount of virus in a viral vaccine. The doses described herein may refer to the total amount of antigen delivered to the subject in the vaccine composition, such as the amount of the vaccine antigen that is delivered to the subject. For example, where the vaccine is an influenza vaccine comprising HA antigen, the doses described herein may refer to the amount of HA antigen in the vaccine composition or the amount of HA antigen delivered to the subject.

Where the vaccine dose is mentioned in µg antigen, typically this is total antigen of the pathogen, more typically it is the amount of a specific antigen, typically the specific antigen is a surface antigen of the pathogen, typically the surface antigen binds to a receptor on target cells, typically the antigen is a haemagglutinin molecule, typically the haemagglutinin is influenza haemagglutinin (HA) antigen, typically the HA is influenza A HA antigen.

For influenza, a typical dose of vaccine has previously been proposed as at least 15 µg haemagglutinin (HA) antigen per strain (WHO Technical Reports Series Number 927, 2005, Recommendations for the production and control of influenza vaccines (inactivated) page 121, Section A5.3 Haemagglutinin content, “at least 15 µg”). For influenza, 15 µg HA is equivalent to approximately 5 × 10³ haemagglutination units (HAU). For a good preparation of live virus, e.g. egg grown PR8 influenza virus, 1 HAU may be equivalent to approximately 10⁵ - 10⁶ plaque forming units (PFU), therefore 15 µg can be approximately equivalent to 5 × 10⁸ - 5 × 10⁹ PFU.

Yam et al., 2015, Front Immunol 6, 207e, did not look at immune protection but they looked at HAI titres as a surrogate marker for protection following i.m. (intra-muscular) immunisation. They showed that repeated doses (x2) of HA antigen delivered i.m. in mice generated HAI titres without adjuvant at 0.03 µg HA antigen but not less per dose. With adjuvant, HAI titres were generated at 0.003 µg HA antigen but not less per dose.

Therefore based on Yam et al., doses below or significantly below a dose (known or) predicted to be effective, i.e. doses for use in the present invention, include, for the delivery of HA antigen or another vaccine of the invention by the i.m. route (a parenteral route) without adjuvant, are doses typically less than 0.03 µg or less than 0.01 µg, or less than 0.003 µg, less than 0.001 µg, or less than 0.0003 µg. Similarly, with adjuvant doses below or significantly below a dose (known or) predicted to be effective, i.e. doses for use in the present invention, include, for the delivery of HA antigen by the i.m. route with adjuvant, are doses typically less than 0.003 µg, less than 0.001 µg, or less than 0.0003 µg, or less than 0.00003 µg.

Thus, a vaccine of the invention, such as a vaccine intended to prevent or treat a respiratory or airborne infection, such as an influenza vaccine, may be administered in a dose of less than 0.003 µg or less than 0.0003 µg, or less than 0.00003 µg. The dose may be, for example, 0.000001 µg to 0.003 µg, 0.00001 µg to 0.0003 µg, 0.0000001 µg to 0.003 µg, or 0.0003 µg to 0.0001 µg of vaccine antigen. Such a dose may be administered with adjuvant or without adjuvant. Such a dose may be administered via any route described herein, including the i.m. route as described by Yam et al, and the i.pul. route as described in more detail herein. The vaccine may be provided to the subject in a single administration, or as two or more administrations. Any one administration of the vaccine may be in an amount as described herein, for example, a first dose of the vaccine may contain such an amount. Alternatively, the total amount of vaccine provided across multiple doses may be an amount as described herein.

Lui et al. (2012, PLoS ONE 7 (12) e52135) found that doses of 1 µg HA antigen delivered x 2 without adjuvant by the i.n. or i.pul. routes were ineffective in protecting against challenge, however the same dose i.m. did provide immune protection. With adjuvant 1 µg HA antigen delivered x2 was effective by the i.n. and i.pul. routes. Noteably antibody at the mucosal surfaces (nasal and lung wash), the site of infection, did not correlate with immune protection.

Although Edwards (WO 2008/025095) did not look at protection, they looked at HAI titre as a potential surrogate marker for protection in sheep. They found that 3 doses of HA antigen delivered i.pul. failed to generate HAI activity without adjuvant at doses of 0.04 µg HA antigen. With adjuvant HAI titre was obtained at 0.04 µg HA antigen but not less per dose.

In Example 1 below a mere 500 PFU of influenza generated immune protection (approximately 0.000064 µg). Therefore, doses less than described in Lui and Edwards are effective by the i.pul. route (a mucosal route). For example, for a vaccine of the invention, such as an influenza vaccine as described herein without adjuvant typically doses less than 1 µg, less than 0.1 µg, less than 0.04 µg, less than 0.01 µg, and/or less than 0.001 µg antigen. With adjuvant typically doses less than 0.04 µg, or less than 0.008 µg, or less than 0.004 µg, or less than 0.001 µg, or less than 0.0004 µg antigen, such as influenza HA antigen.

Thus, a vaccine of the invention, such as a vaccine intended to prevent or treat a respiratory or airborne infection, such as an influenza vaccine, may be administered in a dose of less than 0.001 µg or less than 0.0004 µg. Such a dose may be administered with adjuvant or without adjuvant. Where the dose is administered without adjuvant, a higher dose may be used, such as a dose of less than 0.04 µg, less than 0.01 µg, and/or less than 0.001 µg antigen. Where the dose is administered with adjuvant, a lower dose may be provided, such as less than 0.001 µg, or less than 0.0004 µg antigen, or less than 0.00007 µg, or less than 0.00004 µg, or less than 0.000004 µg antigen. Such a dose may be administered via any route described herein, including the i.pul. route as described in Liu and Edwards. The vaccine may be provided to the subject in a single administration, or as two or more administrations. Any one administration of the vaccine may be in an amount as described herein, for example, a first dose of the vaccine may contain such an amount. Alternatively, the total amount of vaccine provided across multiple doses may be an amount as described herein.

Typically, the vaccine dose is less than 3 × 10⁻², 3 × 10⁻³, 3 × 10⁻⁴, 10⁻⁴, 7 × 10⁻⁵, 6.4 × 10⁻⁵, 3 × 10⁻⁵, 3 × 10⁻⁶, 3 × 10⁻⁷ µg of antigen. Typically, the vaccine dose is greater than or equal to 3 × 10⁻⁸ µg, 3 × 10⁻⁷, 15 × 10⁻⁷, or 3 × 10⁻⁶ µg. The vaccine dose may be greater than or equal to 10⁻⁸, 10⁻⁷, 10⁻⁶ µg of antigen. The vaccine dose may be greater than or equal to 1.28 × 10⁻⁷, 1.28 × 10⁻⁶, 1.28 × 10⁻⁵ or 6.4 × 10⁻⁵ µg of antigen. The dose may be in a range from any of these lower limits to any of the upper limits described herein.

Fernando et al., (2010, PlosONE 5(4) e10266) studied delivery of seasonal trivalent vaccine using a skin patch, Nanopatch™, in mice. They found that 34 ng (0.034 ng) HA antigen delivered by this route gave effective protection against mouse-adapted PR8 virus challenge.

Example 1 below shows much lower levels of antigen were effective (when delivered three times). Therefore, doses less than 34 ng, typically less than 10 ng, typically less than 6.5 ng, typically less than 1 ng, typically less than 0.1 ng, typically less than 0.01 ng, may be used via the skin patch route.

Furuya et al., (2010, J Gen Virol 91, 1450-1460) studied intranasal delivery of inactivated virus. They found that a single dose of 1.6 × 10⁷ PFU (inactivated) was effective at protecting against challenge as measured by weight loss if the virus had been inactivated with γ-irradiation, but similar doses were less effective if the virus had been inactivated with either UV or formalin. Three doses of formalin inactivated virus gave a “collective dose equivalent” to one dose of γ-radiation-inactivated virus based on HAU and this gave similar protection to the γ radiation inactivated virus.

Example 1 below shows that levels far below 1.6 × 10⁷ PFU were effective: 3 doses of 500 PFU gave significant protection against influenza challenge based on weight loss. Thus, a vaccine of the invention, such as a vaccine intended to prevent or treat a respiratory infection, such as an influenza vaccine, may be administered in a dose of less than 1.6 × 10⁷ PFU, less than 10⁷ PFU, less than 5 × 10⁶ PFU, less than 10⁶ PFU, typically less than 10⁵, typically less than 10⁴ PFU, typically less than 5 × 10³ PFU, typically less than 10³ PFU etc. Typically, the vaccine dose is less than 10⁷, 10⁶, 10⁵, 10⁴, 10³, 5 × 10², and/or 10² PFU or CFU, or less than the equivalent of 10⁷, 10⁶, 10⁵, 10⁴, 10³, 5 × 10², and/or 10² PFU or CFU of HA antigen. Typically, the dose is greater than 1 PFU, greater than 10 PFU, greater than 50 PFU, greater than 100 PFU, greater than 200 PFU, greater than 300 PFU, greater than 400 PFU or greater than or equal to 500 PFU. Typically, the vaccine dose is greater than or equal to 10 infectious units (IU), 100 IU, 500 IU, or 1000 IU.

The dose may be, for example, 1 to 1.6 × 10⁷ PFU, 10 to 1,000,000 PFU, 50 to 100,000 PFU, 100 to 25,000 PFU or 200 to 1000 PFU. The dose may be about 500 PFU, 500 PFU ± 100 PFU or 500 PFU ± 50 PFU. Such a dose may be administered with adjuvant or without adjuvant. Such a dose may be administered via any route described herein, including the i.pul. route as described in Liu and Edwards. Where the dose is administered i.pul. in air, it may be provided in an amount of less than 1000 PFU/litre air, less than 500 PFU/litre air, less than 300 PFU/litre air or less than 250 PFU/litre air. The vaccine may be provided to the subject in a single administration, or as two or more administrations. Any one administration of the vaccine may be in an amount as described herein, for example, a first dose of the vaccine may contain such an amount. Alternatively, the total amount of vaccine provided across multiple doses may be an amount as described herein.

In Example 1, the particle to PFU ratio was 1543, assuming 1 RNA copy = 1 virus particle; this value may vary from preparation to preparation. Furuya does not report the number of particles per PFU, however based on the ratio in Example 1 the dose of 1.6 × 10⁷ PFU is about 2.47 × 10¹⁰ influenza virus particles, much higher than the value of 7.7 × 10⁵ virus particles used in Example 1. Therefore, using this worked example, doses of, typically influenza, virus typically less than 2.47 × 10¹⁰ virus particles, less than 10¹⁰ virus particles, less than 10⁹ virus particles, less than 10⁸ virus particles, less than 10⁷, less than 10⁶ virus particles, less than 7.7 × 10⁵ virus particles etc. may be used in the present invention. For example, a suitable dose of a viral vaccine of the invention may contain 10¹ to 10¹⁰ viral particles, from 10⁴ to 10⁸ viral particles or from 10⁵ to 10⁷ viral particles. Such a dose may be administered with adjuvant or without adjuvant. Such a dose may be administered via any route described herein, including the i.pul. route. The vaccine may be provided to the subject in a single administration, or as two or more administrations. Any one administration of the vaccine may contain a number of viral particles as described herein, for example, a first dose of the vaccine may contain such a number of viral particles. Alternatively, the total amount of vaccine provided across multiple doses may contain an amount of viral particles as described herein. Similarly, this relates to inactivated whole virus vaccines measured in PFU (prior to inactivation) or in µg of HA antigen.

To summarise and simplify, based on the above publications, a “mini dose” may be, via a parenteral route without adjuvant less than 0.03 µg and with adjuvant less than 0.003 µg antigen, and via a mucosal route without adjuvant less than or equal to 1 µg antigen and/or less than the equivalent of 1.6 × 10⁷ PFU and with adjuvant less than 0.04 µg antigen and/or less than the equivalent of 1.6 × 10⁷ PFU.

Typically, the dose is a “mini dose”. Typically, the dose is an effective dose.

An effective dose is a dose that provides immune protection against infection and/or disease, for example reduces weight loss and/or other clinical symptoms. Suitable clinical symptoms may be determined by a medical professional depending upon the pathogen and/or disease. For example, for many respiratory infections such as influenza, clinical symptoms may include weight loss, sneezing, nasal congestion, nasal discharge and loss of appetite. Clinical endpoints that may be assessed include hospitalisation, presence of symptoms, duration of illness or symptoms, severity of illness or symptoms and death. An effective dose may be an amount that is effective if delivered on 3 (or more) occasions.

The advantages of using the vaccine preparation include vaccine sparing. That is, much lower amounts of vaccine preparation are required. Put another way, for the same amount of vaccine more people can be treated and more doses can be given. The benefits of this include facilitating more rapid availability of the vaccine in the case of a new disease or pandemic, reduced costs, reduced practical or logistical issues such as storage (especially where temperature controlled storage is required) and transport. The latter are recognised as important by the WHO for developing countries where temperature controlled storage may be limited or affected by unreliable power supplies. The delivery of the vaccine is also practical in the field, not requiring sharp needles for example. Also, by delivering to mucosal surfaces, mucosal immunity is generated at the site of transmission of infection which may be more effective and protective than immunity generated by for example intra muscular immunisation (e.g. Furuya et al., 2010).

Clearly where the worked example is in µg of HA antigen per dose this is readily applicable to other influenza viruses. Similarly, it is clearly applicable to vaccines for other viruses when addressing quantities of antigen in µg. Furthermore, µg of antigen apply to vaccines for other pathogens including bacteria. Calculations based on µg antigen per dose can be used to convert to numbers of virus or other pathogen particles.

Where a virus is similar in size to influenza virus, doses based on virus particle number can be readily applied, for example orthomyxoviruses, paramyxoviruses and coronaviruses.

“Equivalent of x PFU” is used to refer to the equivalent of x PFU influenza. For example, the same number of PFU for viruses of similar sizes, or that number of PFU that have been inactivated in some way such as by UV, formalin or β-propriolactone. If the ratio of PFU to particle number and/or the particle size is different than a suitable calculation/adjustment may have to be made. “Equivalent” refers to delivering the equivalent amount of antigen to influenza A, typically measured variously in µg, PFU and/or particle number, but when measured in PFU and/or particle number typically referring to a vaccine that is derived from whole pathogen typically without significant purification of any particular antigen from the pathogen particle. Any of the doses quoted above in PFU may be the dose of the vaccine to be administered or may be the equivalent PFU.

### Inactivated and Live Vaccines

The vaccine of the invention may comprise live pathogen, such as live virus or attenuated pathogen. The vaccine of the invention may comprise inactivated pathogen such as deactivated or killed virus.

Although most vaccines consist of dead material, some consist of attenuated live bacteria or viruses. Typical attenuated vaccines comprise live pathogens, such as live viruses, that have been cultured under conditions that reduce or disable their virulent properties. Because these live attenuated microorganisms can replicate in the host, and antigen is manufactured in vivo, the actual dose in the host is greater than that physically delivered. Live attenuated viruses have been delivered by routes other than by injection. For example, live attenuated influenza vaccines (LAIV; e.g. Flumist^{tm} or Fluenz^{tm}) can be delivered to the nasal cavity (intra nasal immunisation) at 10⁷ tissue culture infectious dose 50 (TC ID50) per strain.

The vaccine may be an inactivated vaccine. Typically, the vaccine is a straightforward vaccine composition of, typically (UV or formalin, or similar) inactivated virus, without the need for specialised uptake technologies or complex delivery systems such as microencapsulation. The vaccine may be whole pathogens that are inactivated and may comprise the intact pathogen and/or fragments. The Vaccine may comprise virus particles, or the equivalent in virus sub-units, split virions, recombinant protein, synthetic peptides or other antigen formulation. The vaccine may comprise a non-replication competent vector, e.g. adenovirus. However, typically the vaccine is a dead vaccine. Typically, the vaccine is not a live attenuated vaccine.

The vaccine of the invention may be a vaccine that is not capable of productive infection. The vaccine may be a vaccine that is not a live attenuated vaccine and/or not a replication competent vector vaccine and/or not a replication incompetent vector vaccine.

Where the vaccine is produced in situ as part of a method as described herein, some viable pathogen may be delivered as a contaminant. In such an embodiment the method will typically sterilise or deactivate at least 90%, 95%, 99%, 99.9% or 99.99% of the pathogen in the fluid being delivered to the subject, such as the fluid delivered to the subject by a device. The amount of viable or live pathogen will typically be no more than 10%, no more than 5%, no more than 1%, no more than 0.5%, no more than 0.1%, no more than 0.01% or no more than 0.001% of the total amount of the pathogen delivered to the subject.

The whole pathogen may be inactivated gently. Typically, the vaccine will be inactivated with βpropriolactone, ultra violet light (UV), formalin and/or gamma irradiation. If inactivated using UV then typically it will be inactivated with less than 10,000 Jm⁻² UV, more typically less than 1,000 Jm⁻², more typically less than or equal to 600 Jm⁻². Typically, the dose of UV is less than 200 Jm⁻², typically 5-200 Jm⁻², and/or typically greater than 200 Jm⁻².

An inactivated vaccine may retain antigenic properties that allow an immune response against the vaccine to be achieved in the subject that is treated with the vaccine. However, one or more other properties of the pathogen are removed or reduced. For example, an inactivated vaccine may comprise a pathogen that has reduced infectivity or reduced reproductive capability compared to the equivalent pathogen that is not inactivated. Typically, the activity of the pathogen, such as its infectivity or reproductive capability is reduced to 90% or less, 80% or less, 70% or less, 60% or less, 50% of less, 40 % or less, 30% or less, 20% or less, 10% or less, 5% or less, 2% or less, 1% or less, 0.5% or less, 0.1% or less, 0.01% or less, 0.001% or less or 0.0001% or less of the activity of the pathogen prior to inactivation, or of wild type pathogen. The pathogen may be inactivated as part of a method as described herein, for example by using a device that inactivates the pathogen prior to delivery to the subject. The pathogen may be provided in an inactivated form for use in accordance with the present invention. The virus or other pathogen may be inactivated with γ-irradiation, UV, formalin and/or β-propriolactone, or by another means.

### Aerosols

Typically, the vaccine will be delivered to the lungs. Typically, the vaccine will be in the form of and/or be delivered as an aerosol.

For practical purposes aerosol particles of different sizes behave differently: to be a true aerosol where the particles do not settle out requires particle size to be less than 10 µm. For practical purposes, aerosols with particle diameters of 10 µm or less may be delivered to the lungs. Aerosols with particle sizes greater than 10 µm diameter may be delivered to the nasal passageways (intra nasal delivery or immunisation) rather than to the lungs. Confidence that particles are delivered to the lungs is improved if the particle size is significantly less than 10 µm in diameter.

Typically, the vaccine (antigen and/or inactivated pathogen) is in particles of less than 10 µm, or less than 5 µm, or less than 3 µm, or less than 1 µm in diameter. More typically the aerosol will have a mean particle size of less than or equal to 10 µm diameter, more typically less than or equal to 6 µm diameter, more typically less than or equal to 3 µm diameter, more typically 3 µm ± 3 µm diameter, more typically a count median aerodynamic diameter of 0.1 -2.5 µm, and/or a mass median aerodynamic diameter of 0.2 - 5.0 µm, more typically approximately a count median aerodynamic diameter of 0.72 - 0.78 µm, and/or a mass median aerodynamic diameter of 1.3 - 1.5 µm. Typically, the particles will have a count median aerodynamic diameter of less than 5 µm, or less than 3 µm, or less than 1 µm and/or a mass median aerodynamic diameter of less than 5 µm, or less than 3 µm, or less than 1 µm. These particle sizes are typically used where the vaccine is to be delivered to the lungs, i.e. i.pul administration, such as by inhalation.

Typically, the vaccine will comprise an aerosol with a particle size of ≤10 µm, typically administered to the lungs, typically of a dose of 3 × 10⁻⁸ to 3 × 10⁻⁴ µg and/or 10 to 10⁶ IU; typically this dose will be delivered 3 times or more.

### Pre-Prepared and in situ Vaccine Production

Vaccines as described herein may be manufactured in advance and prepared so that known doses can be delivered to subjects. Such vaccine preparations that are manufactured in advance and are provided for subsequent administration to a subject are defined as “manufactured vaccine preparations” or “pre-manufactured vaccine preparations” in this application.

Typically, the vaccine used in the invention is a pre-manufactured vaccine preparation. Typically, a pre-manufactured vaccine of the invention is not capable of productive infection.

Such a pre-manufactured vaccine preparation may be combined with a device as discussed below, such as a nebuliser. This combination may then produce an aerosolised vaccine for use in therapy, typically the treatment or prevention of an infectious disease.

In another embodiment of the invention, the vaccine may be produced in situ as part of the methods of the invention. For example, a device may be used that prepares a suitable vaccine composition and delivers that composition to the subject. In some embodiments, the methods of the invention comprise inactivating live pathogens in order to produce an inactivated vaccine composition of the invention. Typically, such methods inactive or sterilise at least 90%, at least 95%, at least 99%, at least 99.9% or at least 99.99% of the live pathogens before delivery to the subject.

### Devices for Use in the Invention

The vaccine may be delivered to the subject using a device. In some embodiments, the vaccine is prepared in situ as part of the method of the invention.

In some embodiments, the device is a device that is capable of delivering air or other fluids to the respiratory system of the subject. The device may deliver air or other fluid for the subject to breathe in or out.

The device may be a respiratory device, such as a device that delivers the air or other fluid directly to the respiratory system of the subject, such as into the mouth or nasal cavity, into the airways or into the lungs of the subject. Examples of such devices include ventilators and face masks. The device may comprise a mask to be placed over the nose and mouth of the subject. The device may comprise a tracheostomy tube or nasal cannula.

The device may comprise means for delivering the vaccine to the subject, for example, means for delivering a pre-determined dosage of the vaccine to the subject. In some embodiments, the device comprises a nebulizer. The device may allow the vaccine to be delivered to the subject, such as by the subject breathing air containing the vaccine through the device.

The device may be a device that processes air that is to be breathed by a subject. For example, the device may be an air processing device or apparatus such as an air conditioning system or a ventilation system such as a room or building ventilation system. The device may be an air sterilisation device. The device may produce the vaccine in situ and provide the vaccine in air that is to be breathed by the subject.

Previously, the inventors have described an innovative device that provides personal protection against airborne pathogen (WO2008/120005), incorporated herein by reference. Instead of a filter it inactivates the airborne pathogen using UV light, thus overcoming the problems of leakage, wearability and blockages. An important further aspect of this is that inactivated pathogen may be delivered to the user, potentially immunising the user.

It is now recognised that a low dose of pathogen could be used to immunise the user. Producing vaccine by inactivating microorganisms in the air is described herein as the vaccine being “made in situ”. Based on the number of droplets produced on coughing (5×10⁵, Lindsley WG et al., 2012, J Occup Environ Hyg 9 (7) 443-449, Quantity and size distribution of cough-generated aerosol particles produced by influenza patients during and after illness) the dose received using the device could be substantial if such an aerosol was inhaled, especially if each droplet contained a number of pathogens. However, the numbers of pathogens in the air more generally are much lower than this (Yang W., Elankumaran S. and Marr L.C., 2011, J R Soc interface 8, 1176-1184.). Hence it would have been assumed that the device could not be used in general environments to immunise.

As vaccines typically contain 15 µg antigen (see above), it would have been assumed that the device could only be used to immunise where the level of pathogen in the air was sufficient to deliver a similar amount. It would have been assumed that the device could not be used in general environments to immunise.

Nevertheless, the inventors investigated whether it was possible to immunise at very low doses that might be encountered if the device were to be used in more general environments. This is described in Example 1 below.

In accordance with the present invention, a respiratory device, such as the device described in WO2008/120005, may be used to deliver a vaccine to the user, and to thereby achieve a protective effect.

In some embodiments the device inactivates some or all of the pathogens that enter the device before they are delivered to the subject. As discussed above, the output from the device to the subject may therefore be or may comprise an inactivated pathogen, such as an inactivated virus. The device may be used to inactivate pathogens that are specifically introduced into the device, such as to produce an inactivated vaccine in situ from an active pathogen. The device may be used to inactivate pathogen in the surrounding air, for example by inactivating pathogen that is breathed through the device by the user, thereby reducing the active pathogen from the surrounding air that reach the subject. The device may inactivate all of the relevant pathogen before delivering to the subject, or may deactivate substantially all of the pathogen. The device may allow some active pathogen to reach the subject, such as up to 1%, up to 2%, up to 5% or up to 10% of the live pathogen in the surrounding air, or the live pathogen that enters the device.In some embodiments of the invention, the vaccine may therefore be produced in situ from live microorganisms by inactivating them in the air. A typical device for generating the vaccine in situ comprises a chamber into which air is received, UV from a UV source in that chamber that inactivates the pathogen in the air, allowing discharged air to comprise inactivated pathogen; typically, the chamber is in fluid communication with a face mask, and typically the device is portable. Typically, the sterilised air is delivered from the chamber to the mask, protecting the wearer of the mask. Alternatively, or in addition, the air may be received from the mask into the chamber, allowing protection of the receiver of the air from pathogens breathed out by the wearer. There is provided therefore a combination of the air sterilising device above and the vaccine produced in situ by the device. Typically, the vaccine is breathed in. Typically, the vaccine delivered to the lungs.

When using UV to inactivate pathogen in situ, doses of UV delivered to the air and/or pathogen include less than 10,000 Jm⁻², more typically less than 1,000 Jm⁻², more typically 600 ± 300 Jm⁻², typically less than 600 Jm⁻², typically greater than 200 Jm⁻², and/or typically 5-200 Jm⁻².

Typically, the subject to be treated is wearing the device and/or is in the environment of the person wearing said device for generation the vaccine in situ.

The vaccine is typically predefined. That is the disease for which the vaccine is intended to be used against is known and the vaccine is selected to be used for that diseases. Live organisms such as bacteria or viruses may be delivered into the entrance of the chamber, for example as an aerosol, prior to treatment with UV in the chamber. They may be provided as a culture of the organisms or as isolated or purified organisms.

Typically, the device is used in situations where the dose received on inhaling (breathing in) treated air is significantly less than previously identified, known or predicted to be effective doses of vaccines for that pathogen, and where the vaccine is or may be delivered to the lungs.

As seen above, the data from Furuya (2010) indicate that for whole inactivated pathogens (in this case influenza), including inactivated with UV, when delivered to the airways (i.n.) to immunise required at least 1.6 × 10⁷ PFU. This is equivalent to about 2 µg HA antigen, if the particle to PFU ratio was similar to that in Example 1 below. This is consistent with Lui, 2012, who found that without adjuvant, i.pul. immunisation x 2 was not effective at 1 µg HA antigen.

Yang et al., 2011, see above, measured IAV in a health care centre, a day care centre and on aeroplanes. The average level was 51 PFU m⁻³, up to 119 PFU⁻³. Based on an adult breathing rate of 20 m³d⁻¹, this equates to doses during exposures of 1, 8 and 24 hours of 43, 342 and 1026 PFU (up to 99, 791 and 2374 PFU).

Blachere et al., (2009, Clin Infect Dis 48, 438-440) found a range of 800-27,500 PFU m⁻³ in a hospital emergency department giving doses of 667-23,000, 5333-183,000 and 16,000-550,000 PFU during exposures of 1, 8 and 24 hours respectively.

In Example 1 below, 500 PFU (the preparation has 1543 particles per PFU, therefore this was 7.7 × 10⁵ particles, or about 6.4 × 10⁻⁵ µg) produced immune protection.

According to the present invention therefore it has now been recognised that the vaccine may be made in situ, and/or a protective device may be used, in environments including health care centres, day care centres and on aeroplanes, and in other environments were an effective dose may be delivered to the recipient of the treated air and where the dose is less than or much less than that previously thought to be required to achieve an immunising effect, such as less than 1.6 × 10⁷ PFU, for example around 500 PFU. The device may be used by a subject who is at an increased risk of infection, such as a subject in an environment that provides an increased risk of respiratory or airborne infection. A device as described herein may be used to inactivate a pathogen from the air, and to provide the subject with air containing substantially no active pathogen, or a reduced amount of said pathogen, and said air containing inactivated pathogen produced by the device. For example, a subject who is in an environment known or expected to include an airborne pathogen such as a respiratory infectious pathogen in the air, may use such a device to reduce their exposure to the live pathogen and to achieve a protective or therapeutic effect against the pathogen.

These environments typically include indoor areas such as schools, hospitals, surgeries, at home, care homes, theatres, cinemas, fitness centres, gyms, and shops, and modes of transport such as aeroplanes, buses, coaches, trains, taxis, cars, boats and cable cars.

The invention therefore provides a method of preventing or treating infection by airborne infectious agents, the method comprising inactivating infectious agents in the air, and providing the air comprising the inactivated agents to a subject. The inactivation may be achieved by any of the methods described herein, such as by treating the air comprising infectious agents with UV or formalin. Typically, the dose of agent administered to the subject in this method is a dosage as described above. Typically, the dose of inactivated agent administered to the subject is a dosage as described above. Typically, the air comprising inactivated agent is administered to the lungs of the subject, via i.pul. administration, such as by inhalation. Typically, the airborne infectious agent is a respiratory pathogen such as a respiratory virus as described above, such as an orthomyxovirus, typically an influenza virus such as influenza A.

The vaccine produced in situ and/or pre-manufactured vaccine preparation may be delivered on one occasion, greater than one occasion, greater than two occasions, three occasions, greater than three occasions; the occasions may be on the same day, different days, greater than one day apart, greater than one week apart, 9 or 10 days apart, at least 9 days apart, apart by less than a year, or apart by some other separation(s).

The individual occasions or immunisations may be spread out over a period of time, for example over a period of 10 minutes, 1 hour, 2 hours, 3 hours or 10 hours, and may include multiple breaths.

The advantages of the invention in relation to in situ generation of the vaccine include a range of items. Firstly, it transforms the use of previously described devices, and related devices, allowing them to be used in everyday situations for generating protective immunity. This makes them much more practical. A key advantage of such devices is that such immunisation can be combined with personal protective equipment capabilities. They also can protect, and immunise, against multiple strains or species of pathogens where there are multiple pathogens present in the air.

Secondly, immunisation is available sooner than manufactured vaccine preparations are in the case of pandemics. Other advantages include being usable where the infection is unknown. It may be used where there is no vaccine, or limited amounts of vaccine, available. It may be used with seasonal infections. It may be used in unimmunised people. It may be used in the elderly, or others for whom regular vaccines may not be suitable or may be less effective. It may be used to boost immunity, especially in those for whom their existing immunity, including immunity as a result of previous infection and/or vaccination, is ineffective, poorly effective or not completely effective. It may be used to prime immunity for future boosting.

Furuya et al., (2010, J Gen Virol 91, 1450-1460) reported that both UV treatment and formalin inactivation of influenza virus reduced the haemagglutination activity of the virus more than gamma irradiation (9 fold reduction in haemagglutination activity compared with 3 fold). Previous investigations have found that UV and formalin did not noticeably reduce heamagglutination activity (Goldstein M.A. and Tauraso N.M., 1970, Effect of formalin, β-propriolactone, merthiolate, and ultraviolet light upon influenza virus infectivity, chicken cell agglutination and antigenicity, Applied Microbiology 19, 290-294). When used to immunise mice intranasally, UV and formalin inactivated virus were not as effective as gamma irradiated virus. However, if formalin inactivated virus was given repeatedly so that the cumulative dose was equivalent to the gamma irradiated virus in terms of haemagglutination units, then it was as effective (Furuya et al., page 1451, last three lines). Doses used to immunise were low doses of 1.6 × 10⁷ PFU (that is 1.6 × 10⁷ PFU that had been inactivated and no longer infectious).

Thus, the state of the art teaches that for dead vaccines (a) doses of vaccine greater than approximately 1.6 × 10⁷ PFU or equivalent are required (especially if UV or formalin inactivated), and (b) UV and formalin inactivated microorganism are less effective or ineffective than gamma irradiated microorganism.

### Pre-Exposure, Priming and Boosting

It is a well-recognised feature of acquired immunity that following an initial exposure to antigen resulting in a primary immune response, further exposure to the priming antigen can lead to a secondary immune response which is usually stronger. These immunisations are referred to as priming and boosting respectively. Further exposure to antigen will result in further boosting of the immune response. Vaccination may consist of one or more than one immunisation. Where there are more than one immunisations these can be referred to as a priming dose followed by one or more boosting doses (boosters). For priming and boosting to be effective clearly the antigen needs to be the same for all doses.

Accordingly, the vaccine may be administered in a single dose. Alternatively, administration can involve a first (priming) dose, followed by one or more (boosting) doses. For example, the initial administration may be followed by one or more further administrations. In some embodiments, the further administration(s) are given at hourly, daily, weekly, or monthly intervals. The total effective amount of an agent present in the compositions of the invention can be administered to a subject as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Where one or more doses are administered, each dose may be administered over a period of time, for example over a period of 10 minutes, 1 hour, 2 hours, 3 hours or 10 hours, and may include multiple breaths.

The vaccine may be delivered repeatedly; it may be delivered twice. The vaccine may be delivered three times. The vaccine may be delivered three times or more. The vaccine may be delivered more than three times. In the case of i.pul. delivery, doses less than 3 × 10⁷, less than 10⁷, less than 10⁶, less than 10⁵, less than 10⁴, less than 10³, or less than 10² IU (inactivated) may be used. Likewise doses less than 10⁻³, less than 10⁻⁴, less than 10⁻⁵, less than 10⁻⁶, or less than 10⁻⁷ µg antigen may be used.

Delivery of a single dose at the levels described will stimulate the immune system, as demonstrated in the Example, and can contribute towards immune protection. Delivery of a single dose at this level will variously prime and/or boost an immune response. Typically, the vaccine will be used to prime and/or boost and this may be a homologous and/or heterologous (related and/or unrelated) prime and/or boost. To deliver better immune protection, that is better protection from clinical symptoms, may require multiple doses, typically 3 doses. If the pathogen, and/or antigen being used, is poorly immunogenic, for example if a particular pathogen that is known to be poorly immunogenic, then improved responses may be obtained by variously increasing the number of doses administered, including an adjuvant, and/or using a dose based on the known effective dose e.g. 1 in 10⁴ of the known effective dose by i.m. administration. An alternative would be to use a dose that is higher up one of the ranges specified herein.

### Heterologous Effects

In some cases, priming against one antigen can lead to an enhanced immune response against another on subsequent exposure to the second antigen, or some protection against a different pathogen. This may occur if there is some immune cross-reactivity between the priming antigen and the boosting antigen. For example, it has been noticed that priming with one strain of influenza may lead to protection against another strain of influenza. This is usually where the two strains are closely related. It has also been noticed that, in some species at least, priming via a mucosal route (i.n. or intra pulmonary, i.pul., routes) is more effective than parenteral administration at generating this protection against a different strain (Takada A. et al., 2003, Vaccine 21 (23) 3212-3218; Perrone L.A. et al., 2009, J Virology 83 (11) 5726-5734). This cross-protection is called heterologous protection, in this case against a related pathogen. The mechanism for priming may involve for example cross-reactivity at the level of T cells where priming generates T cells against a shared antigenic epitope which in turn support antibody responses against the challenge pathogen.

In real world situations, individuals are likely to have been previously exposed to pathogens that are related to an infectious threat. For example, most people have been infected with influenza, and/or been vaccinated against influenza. There is some evidence that such previous exposure may enhance future immunisations against similar but different influenza strains. This principle is likely to apply also even to emerging infections such as MERS and SARS (both of which are coronaviruses, and coronaviruses are common infections) as well as to influenza pandemics.

### Heterologous Effects (Unrelated Pathogen)

In some circumstances, immunising against one pathogen results in immune protection against unrelated pathogens (Goodridge HS, et al., 2016, harnessing the beneficial heterologous effects of vaccination, Nature Reviews Immunology 16, 392-400). This is termed a heterologous (unrelated) pathogen.

HAI titre is often used as a surrogate marker of immune protection, even though as seen above (Karlsson 2016) there is not necessarily a correlation, especially where adjuvant is used.

Interestingly, in some model systems even standard protective immunisation had little effect on repeated low dose challenge (Song et al., 2015, Repeated low-dose influenza virus infection causes severe disease in mice: a model for vaccine evaluation, J Virol 89 (15) 7841-51).

One embodiment is a vaccine for use in the treatment or prevention of an infectious disease wherein the dose of vaccine is less than 0.03 µg and/or less than the equivalent of 10⁷ CFU or PFU

Another embodiment is a vaccine wherein the dose is less than 0.003 µg and/or less than 10⁶ CFU or PFU. In another embodiment the vaccine dose is less than 0.0003 µg and/or less than the equivalent of 10⁵ CFU or PFU.

The vaccine may be used in the treatment of a heterologous pathogen (related) and/or a heterologous pathogen (unrelated).

### Personal Protective Equipment

A large number of masks are available with the aim of protecting against airborne material. These devices usually work on the basis of the air passing through a filter. However, it is generally recognised that the effectiveness of filter-based devices for protecting individuals against infection is limited. The primary issue is related to the action of the filter. Filters cause a pressure drop across them. The pressure drop results in leakage of the mask at the seals so that the wearer is still exposed to pathogen by air entering around the mask. Furthermore, this pressure drop opposes breathing and therefore limits the length of time that the devices can be worn. A further problem is caused when the filters become blocked with matter thus causing the pressure differential to be even greater resulting in more leakage into the mask and potential contamination.

In accordance with the present invention, a mask or other personal protective equipment may be a device as described above, such as a device that inactivates pathogen, and delivers inactivated pathogen to the user of the device or to other subjects, such as those breathing air that exits the device. Inactivation of the pathogen may be achieved by any of the methods described here, such as using UV. Such a device may or may not comprise a filter. A typical device may permit the inactivated pathogen to be breathed in by the user of the device. A typical device may deliver the inactivated pathogen to the user for inhalation, such as delivery into a face mask or breathing mask. A typical device may inactivate pathogen that is exhaled by the user of the device. Such a device may inactivate such pathogen and deliver it from the device into the air, such that other subject, such as those nearly or those in the same room or building as the user of the device, are delivered the inactivated pathogen and may receive that inactivated pathogen in the lungs by inhalation.

Personal protective equipment (PPE) and vaccines are the primary ways of interfering with transmission (PPE) and reducing the susceptible population (vaccination).

### BRIEF DESCRIPTION OF THE DRAWINGS

The invention is further described by way of example only with reference to the attached figures:

FIG. 1. Study schematic. Upward pointing arrows show days of nasal wash sampling; (S) show days of serum sampling. Days are numbered relative to the H3N2 priming infection on T=0.

FIG. 2. Serum HAI titres against H3N2 virus for Groups 1-4. Points represent geometric mean titre for each group

FIG. 3. Serum HAI titres against H1N1 virus. Points represent geometric mean titre for each group. Titres of <4 are plotted as 2 to allow visualisation.

FIG. 4. Group mean nasal wash cell counts. Points represent the geometric mean titre for each group.

FIG. 5. Group geometric mean nasal wash titres following H1N1 challenge on day 56. Samples with no plaques detected were plotted as 1 PFU/ml.

FIG. 6. Weight change following H3N2 virus challenge. Points represent group means.

FIG. 7. Weight change following H1N1 aerosol spray. Points represent group means.

FIG. 8. Weight change following H1N1 intra-nasal challenge. Points represent group means.

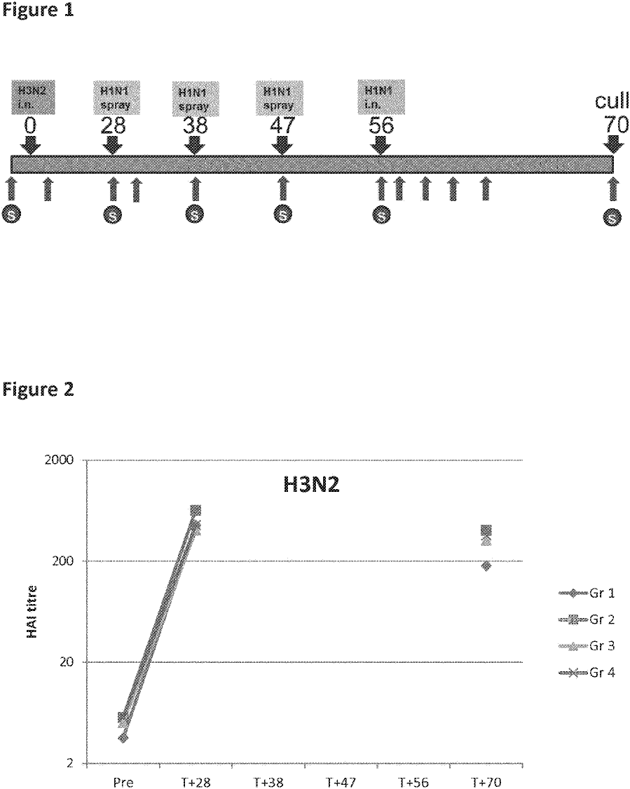

### EXAMPLE 1

When infected by influenza, ferrets share symptoms that are very similar to human influenza. For this and other reasons ferrets are therefore considered perhaps the best animal model for human influenza. Accordingly, ferrets were used to test immunisation with very low doses of influenza. Manufactured vaccine preparation, in the form of formalin killed virus, and in situ vaccine, in the form of live virus passed through a UV sterilising device as described in patent application WO2008/120005, were used. Animals were pre-infected on day 0 with H3N2 virus (Influenza A/Perth/16/09) to mimic the natural situation where most people have already been infected with one or more stains of influenza and/or been vaccinated against seasonal influenza. The immunising and challenge virus was an H1N1 virus (Influenza A/California/04/09).

### Overall Experimental Design

All ferrets were primed by infection with 100 PFU H3N2 virus delivered i.n. at T=0. After 28 days, ferrets were divided into 4 groups for aerosol spray:

TABLE 1

Study groups.

| Group | Aerosol virus | Days of aerosol spray |
|---|---|---|
| 1 | H1N1 untreated | T+28 only |
| 2 | H1N1, UV-inactivated | T+28, 38, 47 |
| 3 | H1N1, formalin-inactivated | T+28, 38, 47 |
| 4 | Mock (PBS) | T+28 only |

H1N1 untreated animals were exposed to H1N1 virus through the sterilisation device but with the UV light off. Therefore, the animals were exposed to live virus.

H1N1, UV-inactivated animals were exposed to H1N1 virus through the sterilisation device but with the UV light on (dose 600 Jm⁻² UV delivered). Therefore, the animals were exposed to UV-inactivated virus.

H1N1, formalin-inactivated animals were exposed to H1N1 virus that had been inactivated with formalin through the sterilisation device but with the UV light off. Therefore, the animals were exposed to formalin-inactivated virus.

Mock (PBS) animals were exposed to PBS through the sterilisation device with the light off. Therefore, the animals were an un-immunised control group, not exposed to virus.

All aerosol immunising doses were of 500 PFU delivered by aerosol to the lungs. The nebuliser delivered particles of count median aerodynamic diameter 0.72 - 0.78 µm, mass median aerodynamic diameter 1.3 - 1.5 µm.

On day T+56, all ferrets were challenged with a low dose (100 PFU) of H1N1 virus i.n.

### Aerosol Infection of Ferrets

Two 5 minute sprays were conducted for each group, 3 ferrets per spray, using the sterilisation device connected to a 6-jet Collison nebuliser, in the order groups 4, 3, 2, 1. On days T+38 and T+47, only groups 2 and 3 were sprayed.

A previous study conducted at Public Health England (PHE) Porton using the Collison nebuliser and Henderson apparatus showed that the presence of the air sterilisation device (with UV light set to off) had no measurable effect on the spray factor of the H1N1 virus. Using the known mean spray factor of 1.53 × 10⁻⁶, and a mean weight (measured on T+26) for the ferrets of 0.9066 kg, it was calculated that a nebuliser concentration of 1.88 × 10⁵ PFU/ml was required to give a presented dose of 500 PFU to each ferret. For formalin-fixed virus, a dilution was used based on the recovery in HAU, and the measured PFU/HAU ratio a ratio of 7.8 × 10⁴ PFU/HAU for the starting material. Thus, on days 28, 38 and 47 the immunising dose was 500 PFU of virus.

### Results

### Priming of Ferrets With H3N2 Virus

Ferrets in all groups were infected intra-nasally with 100 PFU H3N2 virus on T=0. Back-titration of the virus inoculum gave a titre of 375 PFU/ml, which is within 2-fold of the target titre of 500 PFU/ml. Successful infection of all ferrets was confirmed by a rise in nasal wash cell counts on T+4 (see FIG. 4), and rise in haemagglutination inhibition (HAI) titre on T+28 (FIG. 2). For the 6 ferrets showing the lowest rises in nasal wash cell count, plaque assays were performed on the nasal wash fluid, and confirmed all 6 ferrets were actively shedding virus.

### Serum HAI Titres

All sera were treated with 3 volumes of receptor destroying enzyme (RDE) prior to titration to remove any non-specific inhibitors of haemagglutination. Sera from days T=-3 (pre-bleed), T+28 and T+70 were titrated against H3N2 (A/Perth/16/09) and H1N1 (A/California/07/09, antigenically indistinguishable from A/California/04/09) viruses using chicken red blood cells. In addition, sera taken at T+38, T+47 and T+56 were titrated against H1N1 virus.

All titres against both viruses on T=-3 were ≤ 8, and so considered to be sero-negative. All ferrets showed sero-conversion to H3N2, but not H1N1, virus by T+28 (titres ≥ 320). H3N2 titres remained high (≥ 160) until the end of the study at T+70 (FIG. 2).

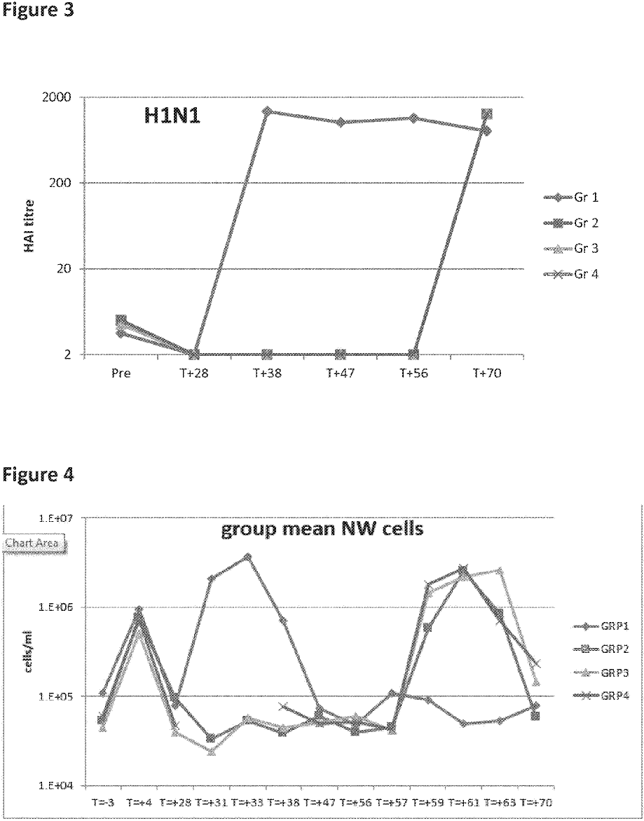

Only group 1 sero-converted to H1N1 virus by 10 days after the aerosol spray on T+28. Groups 2, 3 and 4 sero-converted to H1N1 virus by 14 days after the intra-nasal H1N1 challenge on T+56 (FIG. 3).

### Nasal Wash HAI Titres

Nasal wash fluids taken on T+38 and T+56 were titrated by HAI without prior RDE-treatment, starting from a 2-fold dilution. All titres were ≤ 2 on both days. As these days were presumed to be the most likely to show a mucosal immune response, nasal wash fluids from other days were not titrated.

### Nasal Wash Cell Counts

Counts of viable cells in nasal wash fluid typically rise from ≤ 10⁵ cells/ml to 10⁶-10⁷ cells/ml a few days after virus infection. This rise is a consequence of the innate immune response to infection.

The rise in counts of all groups at T+4 represents the immune response to the H3N2 infection at T=0 (FIG. 4). Cell counts then fell to baseline levels prior to the aerosol infection on T+28. Following aerosol sprays, only group 1 show a rise in cell counts 3-5 days later, suggesting that the UV-treated and formalin-treated viruses were unable to initiate infection of the ferrets. Groups 2-4 show a rise in cell counts 3-7 days after H1N1 virus challenge on T+56, whereas group 1 did not show a rise.

### Titration of Aerosol-Sprayed Virus by Plaque Assay

In order to estimate presented doses of aerosolised virus, remaining nebuliser fluid and collected impinger fluid were titrated by plaque assay. As formalin-fixed virus and virus which had passed through the influenza aerosol sterilising device (IASD) with the UV light on, were expected to show no infectivity, samples of nebuliser and impinger fluids were extracted for RNA and titrated by real-time reverse transcription-polymerase chain reaction (RT-PCR).

TABLE 2

Plaque assay titres of nebuliser and impinger fluids. n/c, plaques not countable. Titres in PFU/ml, mean of 2 replicates. *Two plaques in one well of one replicate.

| | T+28 | | T+38 | | T+47 | |
|---|---|---|---|---|---|---|
| Group | nebuliser | impinger | nebuliser | impinger | nebuliser | impinger |
| 1 | n/c | n/c | | | | |
| 2 | n/c | 0 | 2.63×10⁵ | 0 | 1.03×10⁵ | 2.5* |
| 3 | 0 | 0 | 0 | 0 | 0 | 0 |
| 4 | 0 | 0 | | | | |

Due to technical problems with the Madin-Darby Canine Kidney (MDCK) cells it was not possible to obtain accurate titres for the T+28 samples. Infection was apparent (in n/c groups), but due to problems with the monolayer plaques could not be counted accurately. The group 2 nebuliser titres on T+38 and T+47 were within 2-fold of the target titre of 1.88 × 10⁵ PFU/ml. The same dilution of the same virus stock was used in the nebuliser for groups 1 and 2 on T+28, as for group 2 on T+38 and T+47. The spray factor calculated from RT-PCR titres for Group 1 was very close to the expected value, supporting the conclusion that the target presented dose of 500 PFU per ferret had been achieved. RT-PCR also confirmed that the dose of virus delivered on day T+28 was comparable to those delivered on days T+38 and T+47. No live virus was detected in the formalin-fixed virus group 3. No infectivity was detected in the impinger following UV-treatment of the virus for group 2 at T+28 and T+38. The low titre of 2.5 PFU/ml (calculated from 2 plaques in a single well) seen in the impinger for group 2 at T+47 was only detected in one impinger replicate. As none of the group 2 ferrets showed any signs of infection between days T+47 and T+56, it is assumed that the plaques were the result of contamination, either during collection of the impinger fluids or during set-up of the plaque assays.

### Virus Titrations

Nasal washes from groups 1-3 for T+31 (3 days after first aerosol sprays) were titrated to confirm infection of ferrets by the aerosol route (Table 3).

TABLE 3

Plaque titration of T+31 nasal washes.

| Animal ID | Plaques per ml | Animal ID | Plaques per ml | Animal ID | Plaques per ml |
|---|---|---|---|---|---|
| Group 1 | | Group 2 | | Group 3 | |
| 25004 | 3.50×10³ | 24989 | 0 | 24779 | 0 |
| 25005 | 6.75×10⁴ | 24993 | 0 | 24784 | 0 |
| 25002 | 3.50×10⁵ | 24992 | 0 | 24777 | 0 |
| 24998 | 3.75×10³ | 24995 | 0 | 24778 | 0 |
| 24996 | 2.50×10⁴ | 24782 | 0 | 24781 | 0 |
| 25000 | 3.50×10³ | 24783 | 0 | 24994 | 0 |

All 6 ferrets in group 1 (infected with UV light off) were infected and shedding virus. No virus was detected in nasal wash from ferrets of groups 2 (UV light on) or 3 (formalin-fixed).

### Nasal Wash Cell Counts Following Challenge

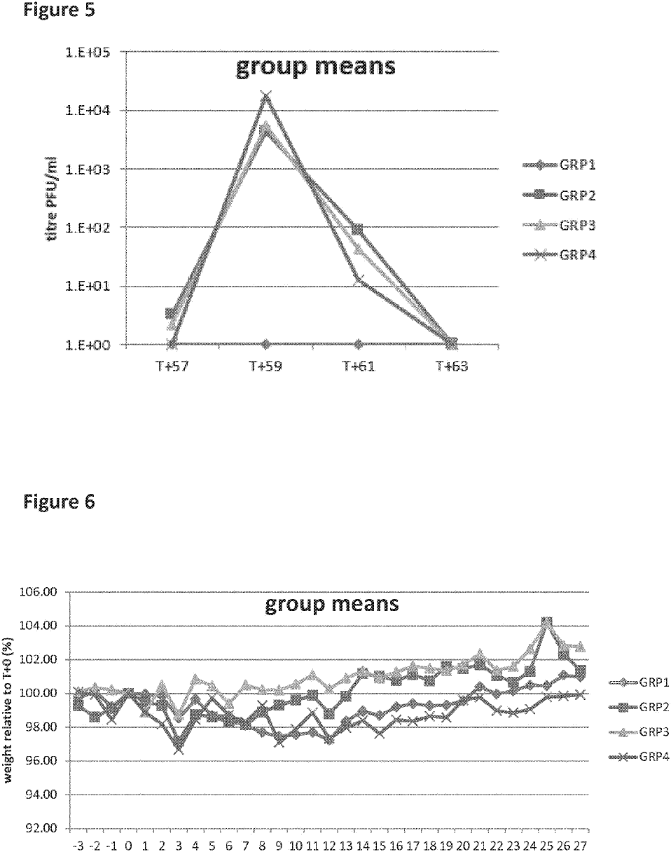

Following H1N1 intra-nasal challenge on T+56, nasal washes were collected and titrated 1, 3, 5 and 7 days later (FIG. 5).

### Virus Titrations Following Challenge

No virus was detected in nasal washes from group 1 at any time-point. All 18 ferrets in groups 2-4 showed virus in nasal washes, peaking 3 days post-challenge. Titres were not significantly different between groups 2-4 on days T+57, T+59 or T+61 (1-way ANOVA). Area under the curve was calculated for each ferret, there was no significant difference between groups 2-4 (1-way ANOVA, p = 0.20).

### Clinical Signs of Infection

Bodyweights were measured daily, except for days T+43-55 inclusive.

The H3N2 virus challenge on T=0 resulted in minor weight loss, with each group showing a dip (1.7 % drop in 1 day) in mean weight on T+3 (FIG. 6).

Other clinical signs of infection (sneezing, nasal discharge and/or loss of appetite) were observed in all groups following the H3N2 challenge, mostly between days 5 and 11 post-infection. No instances of inactivity or diarrhoea were observed in the 28 days post-infection.

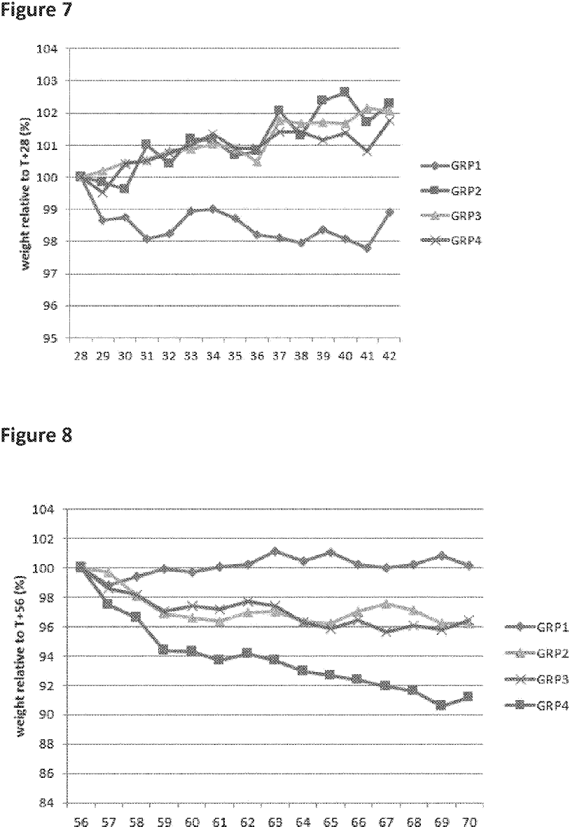

Following the first aerosol sprays on T+28, only group 1 showed weight loss (FIG. 7).

Only group 1 showed any other clinical signs following aerosol spray challenge, namely 5 instances of sneezing. No instances of nasal discharge, inactivity, loss of appetite or diarrhoea were observed in any of the ferrets between days T+28 and T+42.

Following the H1N1 intra-nasal challenge on T+56, significant weight loss was observed in group 4 (mock-sprayed) relative to group 1 (sprayed with infectious virus) (t-test, p < 0.0001 on T+59) (FIG. 8). Maximum mean weight loss of ~10 % (group 4) was in line with previous studies using low-dose intra-nasal H1N1 challenge.

Groups 2 and 3 showed an intermediate level of weight loss relative to groups 1 and 4. Group 2 weights were significantly less than group 1 on days T+58-66 and T+68-70 (t-tests, p < 0.05)(12 out of 14 days). Group 2 weights were significantly greater than group 4 on days T+57-59 and T+61-70 (t-tests, p < 0.05), i.e. 13 out of 14 days.

In order to compare groups across days T+56 to 70 inclusive, weight gain or loss relative to T+56 (i.e. with T+56 set to 0 %) was plotted and area under the curve was calculated for each ferret. Then groups were compared by 1-tailed t-test:

TABLE 4

Comparison of areas under the curves for weight loss between groups. The p-values for each pair of groups are shown.

| | | | |
|---|---|---|---|
| Group | 2 | 3 | 4 |
| 1 | 0.007 | 0.002 | 0.001 |
| 2 | | 0.432 | 0.022 |
| 3 | | | 0.024 |

All groups showed significantly different weight loss from each other, except groups 2 and 3 were not significantly different (Table 4).

There appeared to be no significant difference in observations of sneezing, nasal discharge and loss of appetite between groups 2, 3 and 4 for days T+56 to T+70, although all appeared greater than for group 1.

### Conclusions

H3N2 priming was confirmed by the rise in nasal wash cell counts in all groups, and the high H3N2-specific HAI serum titres seen at T+28. The sprays on T+28 resulted in infection of all ferrets in group 1 (as expected for infectious virus), but no ferrets in groups 2-4, confirming that UV-treatment and formalin treatment had ablated the infectivity of the virus inocula. The spray factor calculated from the RT-PCR titres for group 1 was very close to the expected value, confirming the target presented dose of 500 PFU per ferret, and this was supported by the plaque assay data. Group 1 showed clear protection from the H1N1 virus challenge on T+56: no weight loss, minimal clinical signs, no rise in nasal wash cell count, and no detectable virus in nasal washes. This protection was correlated to the high H1N1-specific serum HAI titre observed at T+56. Groups 2 and 3 showed no detectable HAI titres on T+38 and T+56 in either serum or nasal wash. Groups 2 and 3 were not protected against infection per se (rise in nasal wash cells, virus shedding with peak on T+59, sero-conversion to H1N1), but showed immune protection against disease in terms of reduced weight loss relative to the control group 4.

Thus, aerosol delivery of low doses of (a) formalin inactivated H1N1 virus or (b) in situ generated UV inactivated H1N1 virus three times to the lungs resulted in the generation of immune protection against future challenge with or exposure to infectious H1N1 virus demonstrated by significantly reduced clinical symptoms in particular significantly reduced weight loss.

This immune protection was seen where an HAI titre was not detectable, and therefore below that often taken as required for effective protection.

The immune protection described here is expected to apply to all airborne pathogens; it is expected to apply to all airborne viruses, all airborne RNA viruses, all airborne negative strand RNA viruses, to all orthomyxoviruses, to all influenza viruses and/or to influenza A viruses.

Based on the effective dose of 500 PFU, it can be deduced that doses of similar magnitude or larger stimulate the immune system and will also be effective if delivered by the same route and in repeated doses. It is known that repeat immunising will boost the immune responses therefore based on these observations it is recognised that for significantly lower doses than 500 PFU more than 3 immunisations are likely to be required. Clearly the 500 PFU or similar may be inactivated by UV or formalin or similar inactivating agent, and may be provided as a manufactured vaccine preparation or inactivated in situ.

What is claimed is:

1. A method for the prevention or treatment of a disease in a subject comprising administering an effective amount of a vaccine to the subject wherein the vaccine is not a live attenuated vaccine and wherein the vaccine is used in a dose of, via a parenteral route without adjuvant less than 0.03 µg antigen and with adjuvant less than 0.003 µg antigen, and via a mucosal route without adjuvant less than 1 µg antigen and with adjuvant less than 0.04 µg antigen.

2. The method according to claim 1, wherein the vaccine is used to prevent or treat a respiratory infection and/or airborne infection.

3. The method according to claim 1, wherein the vaccine is administered to the respiratory tract.

4. The method according to claim 1, wherein the vaccine is administered to the lungs.

5. The method according to claim 1, wherein the disease is A a respiratory or airborne infection, wherein the vaccine is delivered to the lungs of the subject to be treated and wherein the vaccine is administered in a dose of less than 0.003 µg.

6. The method according to claim 1, wherein the dose is less than 0.001 µg.

7. The method according to claim 1, wherein the dose is less than 0.0003 µg.

8. (canceled)

9. The method according to claim 1, wherein the infection is a viral infection.

10. The method according to claim 9, wherein the vaccine is or comprises an inactivated virus.

11. The method according to claim 1, wherein the vaccine is a vaccine against influenza, tuberculosis, MERS, SARS, rhinovirus, measles, Ebola, Chlamydia pneumonia, respiratory syncytial virus, pneumococci or FMDV.

12. (canceled)

13. The method according to claim 1, wherein the method comprises producing said vaccine composition and administering it to the lungs of a subject.

14. The method according to claim 13, wherein said method comprises inactivating the infectious agent responsible for said disease and delivering the inactivated agent to the lungs of the subject.

15. The method according to claim 13, wherein the vaccine is made in situ using a portable device.

16. (canceled)

17. (canceled)

18. (canceled)

19. The method according to claim 1, wherein the vaccine is delivered to the subject using a device comprising a nebulizer.

20. The method according to claim 1, wherein the vaccine is delivered as and/or comprises an aerosol.

21. The method according to claim 1, wherein the vaccine does not comprise or is not co-administered with an adjuvant.

22. The method according to claim 1, wherein
(a) the subject to be treated has already been primed by exposure to the same, or similar, pathogen and/or has been vaccinated with a vaccine for the same or similar pathogen; or
(b) the vaccine is delivered in an initial priming dose followed by a boost; or
(c) the vaccine is delivered on 3 or more occasions to a subject.

23. (canceled)

24. (canceled)

25. The method according to claim 1, wherein the disease is an infectious disease, wherein the effective amount is less than 0.003 µg antigen .

26. The method according to claim 3, wherein
(i) the vaccine is made in situ by inactivating live virus in the air, or
(ii) the vaccine is made in situ by inactivating live virus in the air wherein said method is carried out in an indoor environment or in a mode of transport.

27. A method of delivering a vaccine to a human subject at risk of developing a viral infection, wherein the vaccine is a vaccine against said virus that comprises inactivated virus, and is not a live attenuated vaccine, and wherein the vaccine is delivered via a mucosal route in a dose of, without adjuvant less than 1 µg antigen, or with adjuvant less than 0.04 µg antigen, optionally wherein said method comprises inactivating said virus and delivering the inactivated virus to the lungs of the human subject.
